SEQUENCE LISTING (70 sequences)

SEQ ID NO: 1 - protein, 113 aa, Artificial Sequence [sequence omitted]

SEQ ID NO: 2 - protein, 5 aa, Artificial Sequence
  Antibody sequence
NSGMH

SEQ ID NO: 3 - protein, 17 aa, Artificial Sequence
  Antibody sequence
VIWYDGSKRYYADSVKG

SEQ ID NO: 4 - protein, 4 aa, Artificial Sequence
  Antibody sequence
NDDY

SEQ ID NO: 5 - protein, 107 aa, Artificial Sequence [sequence omitted]

SEQ ID NO: 6 - protein, 11 aa, Artificial Sequence
  Antibody sequence
RASQSVSSYLA

SEQ ID NO: 7 - protein, 7 aa, Artificial Sequence
  Antibody sequence
DASNRAT

SEQ ID NO: 8 - protein, 9 aa, Artificial Sequence
  Antibody sequence
QQSSNWPRT

SEQ ID NO: 9 - protein, 440 aa, Artificial Sequence
  Antibody sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  Antibody sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 118 aa, Artificial Sequence
  Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSS

SEQ ID NO: 12 - protein, 7 aa, Artificial Sequence
  Antibody sequence
TYGIGVG

SEQ ID NO: 13 - protein, 16 aa, Artificial Sequence
  Antibody sequence
HIWWNDNKYYSTSLKT

SEQ ID NO: 14 - protein, 8 aa, Artificial Sequence
  Antibody sequence
ISLPYFDY

SEQ ID NO: 15 - protein, 107 aa, Artificial Sequence
  Antibody sequence
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIK

SEQ ID NO: 16 - protein, 11 aa, Artificial Sequence
  Antibody sequence
KASQNVGTAVA

SEQ ID NO: 17 - protein, 7 aa, Artificial Sequence
  Antibody sequence
SASNRYT

SEQ ID NO: 18 - protein, 9 aa, Artificial Sequence
  Antibody sequence
QQYSSYPLT

SEQ ID NO: 19 - protein, 443 aa, Artificial Sequence
  Antibody sequence
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 20 - protein, 214 aa, Artificial Sequence
  Antibody sequence
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 118 aa, Artificial Sequence
  Antibody sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 22 - protein, 5 aa, Artificial Sequence
  Antibody sequence
DSWIH

SEQ ID NO: 23 - protein, 17 aa, Artificial Sequence
  Antibody sequence
WISPYGGSTYYADSVKG

SEQ ID NO: 24 - protein, 11 aa, Artificial Sequence
  Antibody sequence
ARRHWPGGFDY

SEQ ID NO: 25 - protein, 107 aa, Artificial Sequence [sequence omitted]

SEQ ID NO: 26 - protein, 11 aa, Artificial Sequence
  Antibody sequence
RASQDVSTAVA

SEQ ID NO: 27 - protein, 7 aa, Artificial Sequence
  Antibody sequence
SASFLYS

SEQ ID NO: 28 - protein, 9 aa, Artificial Sequence
  Antibody sequence
QQYLYHPAT

SEQ ID NO: 29 - protein, 445 aa, Artificial Sequence
  Antibody sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSASSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 30 - protein, 214 aa, Artificial Sequence
  Antibody sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 120 aa, Artificial Sequence [sequence omitted]

SEQ ID NO: 32 - protein, 5 aa, Artificial Sequence
  Antibody sequence
NYYMY

SEQ ID NO: 33 - protein, 17 aa, Artificial Sequence
  Antibody sequence
GINPSNGGTNFNEKFKN

SEQ ID NO: 34 - protein, 11 aa, Artificial Sequence
  Antibody sequence
RDYRFDMGFDY

SEQ ID NO: 35 - protein, 111 aa, Artificial Sequence [sequence omitted]

SEQ ID NO: 36 - protein, 15 aa, Artificial Sequence
  Antibody sequence
RASKGVSTSGYSYLH

SEQ ID NO: 37 - protein, 7 aa, Artificial Sequence
  Antibody sequence
LASYLES

SEQ ID NO: 38 - protein, 9 aa, Artificial Sequence
  Antibody sequence
QHSRDLPLT

SEQ ID NO: 39 - protein, 447 aa, Artificial Sequence
  Antibody sequence
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 218 aa, Artificial Sequence
  Antibody sequence
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 118 aa, Artificial Sequence [sequence omitted]

SEQ ID NO: 42 - protein, 5 aa, Artificial Sequence
  Antibody sequence
DSWIH

SEQ ID NO: 43 - protein, 17 aa, Artificial Sequence
  Antibody sequence
WISPYGGSTYYADSVKG

SEQ ID NO: 44 - protein, 9 aa, Artificial Sequence
  Antibody sequence
RHWPGGFDY

SEQ ID NO: 45 - protein, 107 aa, Artificial Sequence [sequence omitted]

SEQ ID NO: 46 - protein, 11 aa, Artificial Sequence
  Antibody sequence
RASQDVSTAVA

SEQ ID NO: 47 - protein, 7 aa, Artificial Sequence
  Antibody sequence
SASFLYS

SEQ ID NO: 48 - protein, 9 aa, Artificial Sequence
  Antibody sequence
QQYLYHPAT

SEQ ID NO: 49 - protein, 448 aa, Artificial Sequence
  Antibody sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, Artificial Sequence; identical to SEQ ID NO: 30
  Antibody sequence

SEQ ID NO: 51 - protein, 120 aa, Artificial Sequence
  Antibody sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 52 - protein, 5 aa, Artificial Sequence
  Antibody sequence
SYIMM

SEQ ID NO: 53 - protein, 17 aa, Artificial Sequence
  Antibody sequence
SIYPSGGITFYADTVKG

SEQ ID NO: 54 - protein, 11 aa, Artificial Sequence
  Antibody sequence
IKLGTVTTVDY

SEQ ID NO: 55 - protein, 110 aa, Artificial Sequence
  Antibody sequence
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 56 - protein, 14 aa, Artificial Sequence
  Antibody sequence
TGTSSDVGGYNYVS

SEQ ID NO: 57 - protein, 7 aa, Artificial Sequence
  Antibody sequence
DVSNRPS

SEQ ID NO: 58 - protein, 10 aa, Artificial Sequence
  Antibody sequence
SSYTSSSTRV

SEQ ID NO: 59 - protein, 450 aa, Artificial Sequence
  Antibody sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 216 aa, Artificial Sequence
  Antibody sequence
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 61 - protein, 121 aa, Artificial Sequence
  Antibody sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 62 - protein, 5 aa, Artificial Sequence
  Antibody sequence
RYWMS

SEQ ID NO: 63 - protein, 17 aa, Artificial Sequence
  Antibody sequence
NIKQDGSEKYYVDSVKG

SEQ ID NO: 64 - protein, 12 aa, Artificial Sequence
  Antibody sequence
EGGWFGELAFDY

SEQ ID NO: 65 - protein, 108 aa, Artificial Sequence
  Antibody sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 66 - protein, 12 aa, Artificial Sequence
  Antibody sequence
RASQRVSSSYLA

SEQ ID NO: 67 - protein, 7 aa, Artificial Sequence
  Antibody sequence
DASSRAT

SEQ ID NO: 68 - protein, 9 aa, Artificial Sequence
  Antibody sequence
QQYGSLPWT

SEQ ID NO: 69 - protein, 451 aa, Artificial Sequence
  Antibody sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 215 aa, Artificial Sequence
  Antibody sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0010017 A1
(43) Pub. Date: Jan. 13, 2022

(54) PHARMACEUTICAL COMBINATION OF ANTI-CEACAM6 AND EITHER ANTI-PD-1 OR ANTI-PD-L1 ANTIBODIES FOR THE TREATMENT OF CANCER

(71) Applicants: Bayer Aktiengesellschaft, Leverkusen (DE); Deutsches Krebsforschungszentrum, Heidelberg (DE)

(72) Inventors: Jörg WILLUDA, Glienicke/Nordbahn (DE); Mark TRAUTWEIN, Wülfrath (DE); Rienk OFFRINGA, Heidelberg (DE); Philipp BECKHOVE, Heidelberg (DE); Hans-Henning BÖHM, Heidelberg (DE)

(73) Assignees: Bayer Aktiengesellschaft, Leverkusen (DE); Deutsches Krebsforschungszentrum, Heidelberg (DE)

(21) Appl. No.: 17/293,342

(22) PCT Filed: Nov. 7, 2019
(86) PCT No.: PCT/EP2019/080520

§ 371 Date: May 12, 2021

(30) Foreign Application Priority Data
Nov. 14, 2018 (EP) 18206206.7

(51) Int. Cl. C07K 16/28; A61P 35/00

(52) U.S. Cl. CPC C07K 16/2827; C07K 16/2818; A61K 2039/507; C07K 2317/565; A61P 35/00

(57) ABSTRACT

Pharmaceutical Combination for the Treatment of Cancer The present invention relates to combinations of at least two components, component A and component B for use in the treatment of cancer, component A being an anti-CEACAM6 antibody and component B being an anti-PD-1 antibody, preferentially nivolumab, or pembrolizumab, or an anti-PD-L1 antibody, preferentially atezolizumab, avelumab, or durvalumab.

The present invention relates to combinations of at least two components, component A and component B, component A being anti-CEACAM6 antibody TPP-3310 and component B being an anti-PD-1 antibody, preferentially nivolumab, or pembrolizumab, or an anti-PD-L1 antibody, preferentially atezolizumab, avelumab, or durvalumab.

Another aspect of the present invention relates to the use of such combinations as described herein for the preparation of a medicament for the treatment or prophylaxis of cancer.

Yet another aspect of the present invention relates to methods of treatment or prophylaxis of a cancer in a subject, comprising administering to said subject a therapeutically effective amount of a combination as described herein.

Further, the present invention relates to a kit comprising a combination of:
a components A, being anti-CEACAM6 antibody TPP-3310;
a component B, being an anti-PD-1 antibody, preferentially nivolumab, or pembrolizumab, or an anti-PD-L1 antibody, preferentially atezolizumab, avelumab, or durvalumab, and, optionally
one or more pharmaceutical agents C;

in which optionally either or both of said components A and B are in the form of a pharmaceutical formulation which is ready for use to be administered simultaneously, concurrently, separately or sequentially.

Component A and B preferably are administered by the intravenous route.

In some embodiments the cancer is lung cancer, in particular non-small cell lung cancer (NSCLC), pancreatic cancer, gastric cancer, colorectal cancer, head and neck cancer, bladder cancer, bile duct cancer, breast cancer, cervical cancer, esophageal cancer.

### BACKGROUND TO THE INVENTION

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

T-cell responses against tumor-associated antigens have been described in many tumors and often cause an accumulation of tumor specific memory T cells in lymphoid organs or in the blood. However, the capacity of T cells to react against autologous tumor cells is generally low. Many tumors have the capacity to block effector functions of T cells which contributes to the limited activity of tumor immunotherapy. T-cell unresponsiveness against tumor cells has been demonstrated for a broad variety of cancers.

The immune system depends on multiple checkpoints or “immunological brakes” to avoid over-activation of the immune system on healthy cells. Tumor cells often take advantage of these checkpoints to escape detection by the immune system. CTLA-4 and PD-1 are checkpoints that have been studied as targets for cancer therapy.

Checkpoint proteins regulate T-cell activation or function. Numerous checkpoint proteins are known, such as CTLA-4 and its ligands CD80 and CD86; and PD-1 with its ligands PD-L1 and PD-L2. These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies. Currently, different immunotherapeutic approaches are standing their ground as powerful treatment strategies for a wide range of malignant diseases.

A very prominent and recent example of an outstanding cancer immunotherapy success involves immune checkpoint blockade therapy by monoclonal antibodies (mAb) targeting inhibitory molecules on either immune effector T-cells or antigen presenting cells including tumor cells. Interfering with co-inhibitors has been shown to unleash a powerful antitumor T-cell response (Pardoll: The blockade of immune checkpoints in cancer immunotherapy. Nat Rev Cancer 12: (2012) 252-264).

CTLA-4 has been shown to be aberrantly upregulated and present on the surface of T cells in certain cancers, dampening T-cell activation in response to tumor cells. PD-1 is another immunologic checkpoint that has been found to be upregulated in certain tumors; it inhibits T-cell function contributing to the tumor's ability to evade the immune system.

Antibody blockade of immune checkpoint molecules for immune cell activation and thus for immunotherapy of cancer is a clinically validated approach. In 2011 the CTLA-4 blocking antibody Ipilimumab has been approved by the FDA for the 2nd line therapy of metastatic melanoma (Yervoy). Another example is the blockade of the PD-1/PD-L1 axis for which several drugs are either approved or currently under clinical development and for which impressive clinical responses have been reported in melanoma, lung cancer, RCC, bladder cancer and others (Shen and Zhao: Efficacy of PD-1 and PD-L1 inhibitors and PD-L1 expression status in cancer: meta-analysis. BMJ2018; 362:k3529).

In 2013, a combination of anti-CTLA4 and anti-PD1 mAb treatment was reported to act synergistically in increasing survival and tumor regression in advanced melanoma patients (Wolchok et al.: Nivolumab plus ipilimumab in advanced melanoma. N Engl J Med 369: (2013) 122-133).

CEACAM6 also contributes to the regulation of CD8+ T cell response. In multiple myeloma expressing several CEACAM family members treatment with anti-CEACAM6 mAbs or siRNA silencing CEACAM6 reinstated T cell reactivity against malignant plasma cells indicating a role for CEACAM6 in CD8+ T cell response regulation (Witzens-Harig et al., Blood 2013 May 30; 121(22):4493-503). Very potent anti-CECAM6 antibodies for cancer immunotherapy including TPP-3310 were disclosed in WO 2016/150899 A2.

### Definitions

Unless defined otherwise, all technical and scientific terms used herein have the meaning commonly understood by one of ordinary skill in the art to which this invention belongs. The following references, however, can provide one of skill in the art to which this invention pertains with a general definition of many of the terms used in this invention, and can be referenced and used so long as such definitions are consistent with the meaning commonly understood in the art. Such references include, but are not limited to, Singleton et al., Dictionary of Microbiology and Molecular Biology (2nd ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resource available to the person of ordinary skill in the art providing definitions of terms used herein having the meaning commonly understood in the art can be consulted. For the purposes of the present invention, the following terms are further defined. Additional terms are defined elsewhere in the description. As used herein and in the appended claims, the singular forms “a,” and “the” include plural reference unless the context clearly dictates otherwise. Thus, for example, reference to “a gene” is a reference to one or more genes and includes equivalents thereof known to those skilled in the art, and so forth.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

In accordance with the present invention, the term “antibody” is to be understood in its broadest meaning and comprises immunoglobulin molecules, for example intact or modified monoclonal antibodies, polyclonal antibodies or multispecific antibodies (e.g. bispecific antibodies). An immunoglobulin molecule preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs arranged from amino-terminus to carboxy-terminus e.g. in the following order: FR1, CDR1, FR2, CDR2, FR3, CDR3, FR4.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L-CDR1), 50-56 (L-CDR2) and 89-97 (L-CDR3) in the light chain variable domain and 31-35 (H-CDR1), 50-65 (H-CDR2) and 95-102 (H-CDR3) in the heavy chain variable domain; (Kabat et al., Sequences of Proteins of Immulological Interest, 5th Ed. Public Health Service, National Institutes of Health, Bethesda, Md. (1991)) and/or those residues from a “hypervariable loop” (e.g. about residues 26-32 (L-CDR1), 50-52 (L-CDR2) and 91-96 (L-CDR3) in the light chain variable domain and 26-32 (H-CDR1), 53-55 (H-CDR2) and 96-101 (H-CDR3) in the heavy chain variable domain (Chothia and Lesk; J Mol Biol 196: 901-917 (1987)). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA1, and IgA2. A preferred class of immunoglobulins for use in the present invention is IgG.

The heavy-chain constant domains that correspond to the different classes of antibodies are called [alpha], [delta], [epsilon], [gamma], and [mu], respectively. The subunit structures and three-dimensional configurations of different classes of immunoglobulins are well known. As used herein antibodies are conventionally known antibodies and functional fragments thereof.

An “anti-antigen” antibody refers to an antibody that binds specifically to the antigen. For example, an anti-PD-1 antibody binds specifically to PD-1 and an anti-CECAM6 antibody binds specifically to CECAM6.

The term “specific binding” or “binds specifically” refers to an antibody or binder which binds to a predetermined antigen/target molecule. Specific binding of an antibody or binder typically describes an antibody or binder having an affinity of at least 10⁻⁷ M (as Kd value; i.e. preferably those with Kd values smaller than 10⁻⁷ M), with the antibody or binder having an at least two times higher affinity for the predetermined antigen/target molecule than for a non-specific antigen/target molecule (e.g. bovine serum albumin, or casein) which is not the predetermined antigen/target molecule or a closely related antigen/target molecule. Specific binding of an antibody or binder does not exclude the antibody or binder binding to a plurality of antigens/target molecules (e.g. orthologs of different species). The antibodies preferably have an affinity of at least 10⁻⁷ M (as Kd value; in other words preferably those with smaller Kd values than 10⁻⁷ M), preferably of at least 10⁻⁸ M, more preferably in the range from 10⁻⁸ M to 10⁻¹¹ M. The Kd values may be determined, for example, by means of surface plasmon resonance spectroscopy.

“Functional fragments”, “antigen-binding antibody fragments”, or “antibody fragments” refer to one or more fragments of an antibody that retain the ability to bind specifically to the antigen bound by the whole antibody. “Functional fragments”, “antigen-binding antibody fragments”, or “antibody fragments” of the invention include but are not limited to Fab, Fab′, Fab′-SH, F(ab′)2, and Fv fragments; diabodies; single domain antibodies (DAbs), linear antibodies; single-chain antibody molecules (scFv); and multispecific, such as bi- and tri-specific, antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag).

The term “immunotherapy” refers to the treatment of a subject afflicted with, or at risk of contracting or suffering a recurrence of, a disease by a method comprising inducing, enhancing, suppressing or otherwise modifying an immune response.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

As used herein “CEACAM6” designates the “carcinoembryonic antigen-related cell adhesion molecule 6”, also known as “CD66c” (Cluster of Differentiation 66c), or Non-specific crossreacting antigen, or NCA, or NCA-50/90. CEACAM6 is a glycosylphosphatidylinositol (GPI)-linked cell surface protein involved in cell-cell adhesion. The term “CEACAM6” as used herein includes human CEACAM6 (hCEACAM6), variants, isoforms, and species homologs of hCEACAM6, and analogs having at least one common epitope with hCEACAM6. A reference sequence for human CEACAM6 is available from UniProtKB/Swiss-Prot data base under accession number P40199.3

“Programmed Death-1 (PD-1)” refers to an immunoinhibitory receptor belonging to the CD28 family. PD-1 is expressed predominantly on previously activated T cells in vivo and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” is one of two cell surface glycoprotein ligands for PD-1 (the other being PD-L2) that down regulate T cell activation and cytokine secretion upon binding to PD-1. The term “PD-L1” as used herein includes human PD-L1 (hPDL1), variants, isoforms, and species homologs of hPD-L1, and analogs having at least one common epitope with hPD-L1. The complete hPD-L1 sequence can be found under GenBank Accession No. Q9NZQ7.

As used herein, the terms “patient” or “subject” are used interchangeably and mean a mammal, including, but not limited to, a human or non-human mammal, such as a bovine, equine, canine, ovine, or feline. Preferably, the patient is a human.

### DESCRIPTION OF THE INVENTION

Surprisingly it was observed that a combination of a PD-1 or a PD-L1 immune checkpoint inhibitor and anti-CECAM6 antibody TPP-3310 act more than additive in an in vitro assay performed to evaluate the therapeutic potential of drug combinations for tumor regression.

Therefore the present invention provides combinations of at least two components, component A and component B, component A being anti-CEACAM6 antibody TPP-3310 and component B being an anti-PD-1 antibody, preferentially nivolumab, or pembrolizumab, or an anti-PD-L1 antibody, preferentially atezolizumab, avelumab, or durvalumab.

The combinations comprising at least two components A and B, as described and defined herein are also referred to as “combinations of the present invention”.

Further, the present invention relates to a kit comprising:
a combination of:
   a component A, being anti-CEACAM6 antibody TPP-3310;
   a component B, being an anti-PD-1 antibody, preferentially nivolumab, or pembrolizumab, or an anti-PD-L1 antibody, preferentially atezolizumab, avelumab, or durvalumab, and, optionally
   one or more pharmaceutical agents C;

in which optionally either or both of said components A and B are in the form of a pharmaceutical formulation which is ready for use to be administered simultaneously, concurrently, separately or sequentially.

The invention further provides an anti-CEACAM6 antibody (component A) for use in simultaneous, separate, or sequential combination with an anti-PD-1 antibody or an anti-PD-L1 antibody (component B) in the treatment of cancer, wherein the anti-CEACAM6 antibody comprises the H-CDR1, H-CDR2, H-CDR3, L-CDR1, L-CDR2, and L-CDR3 of antibody TPP-3310.

The invention further provides an anti-CEACAM6 antibody (component A) for use in simultaneous, separate, or sequential combination with an anti-PD-1 antibody or an anti-PD-L1 antibody (component B) in the treatment of cancer, wherein the anti-CEACAM6 antibody comprises the variable heavy chain sequence and a variable light chain sequences of antibody TPP-3310.

The invention further provides an anti-CEACAM6 antibody (component A) for use in simultaneous, separate, or sequential combination with an anti-PD-1 antibody or an anti-PD-L1 antibody (component B) in the treatment of cancer, wherein the anti-CEACAM6 antibody comprises the heavy chain region and light chain region of antibody TPP-3310.

The invention further provides the anti-CEACAM6 antibody TPP-3310 (component A) for use in simultaneous, separate, or sequential combination with an anti-PD-1 antibody or an anti-PD-L1 antibody (component B) in the treatment of cancer, wherein the anti-PD-1 antibody is nivolumab, or pembrolizumab, and the anti-PD-L1 antibody is atezolizumab, avelumab, or durvalumab.

The invention further provides the anti-CEACAM6 antibody TPP-3310 (component A) for use in simultaneous, separate, or sequential combination with an anti-PD-1 antibody or an anti-PD-L1 antibody (component B) in the treatment of cancer, wherein the cancer is lung cancer, in particular non-small cell lung cancer, ovarian cancer, mesothelioma, pancreatic cancer, gastric cancer, colorectal cancer, head and neck cancer, bladder cancer, bile duct cancer, breast cancer, cervical cancer, or esophageal cancer.

The invention further provides the anti-CEACAM6 antibody TPP-3310 (component A) for use in simultaneous, separate, or sequential combination with an anti-PD-1 antibody or an anti-PD-L1 antibody (component B) in the treatment of cancer, wherein at least one of the anti-CEACAM6 antibody, the anti-PD-1 antibody, or an anti-PD-L1 antibody is administered in simultaneous, separate, or sequential combination with one or more pharmaceutical agents (agents C).

The invention further provides a method of treating cancer comprising administering to a patient in need, thereof an effective amount of anti-CEACAM6 antibody TPP-3310 (component A) in simultaneous, separate, or sequential combination with an anti-PD-1 antibody or an anti-PD-L1 antibody (component B).

The invention further provides a method of treating cancer comprising administering to a patient in need, thereof an effective amount of anti-CEACAM6 antibody TPP-3310 (component A) in simultaneous, separate, or sequential combination with an anti-PD-1 antibody or an anti-PD-L1 antibody (component B), wherein the anti-PD-1 antibody is nivolumab, or pembrolizumab, and the anti-PD-L1 antibody is atezolizumab, avelumab, or durvalumab.

The invention further provides the use of anti-CEACAM6 antibody TPP-3310 (component A) for the manufacture of a medicament for the treatment of cancer in simultaneous, separate, or sequential combination with an anti-PD-1 antibody or an anti-PD-L1 antibody (component B).

The invention further provides the use of anti-CEACAM6 antibody TPP-3310 (component A) for the manufacture of a medicament for the treatment of cancer in simultaneous, separate, or sequential combination with an anti-PD-1 antibody or an anti-PD-L1 antibody (component B), wherein the anti-PD-1 antibody is nivolumab, or pembrolizumab, and the anti-PD-L1 antibody is atezolizumab, avelumab, or durvalumab. The components may be administered independently of one another by the oral, intravenous, topical, local installations, intraperitoneal or nasal route.

In accordance with another aspect, the present invention covers the combinations as described supra for the treatment or prophylaxis of cancer.

In accordance with another aspect, the present invention covers the use of such combinations as described supra for the preparation of a medicament for the treatment or prophylaxis of cancer.

Component A of the Combination

Component A is anti-CEACAM6 antibody TPP-3310 which was disclosed in WO 2016/150899 A2. Further anti-CECAM6 antibodies disclosed in WO 2016150899 A2 are for example TPP-3714, TPP-3820, TPP-3821, TPP-3707, and TPP-3705. These antibodies are human or humanized antibodies binding of human CEACAM6 with high affinity, are cross-reactive to monkey CEACAM6, do not bind to any paralogs, especially CEACAM1, CEACAM3, and CEACAM5, and are able to relieve CEACAM6-mediated immunosuppression.

The term “anti-CEACAM6 antibody” relates to an antibody which specifically binds the cancer target molecule CEACAM6, preferentially with an affinity which is sufficient for a diagnostic and/or therapeutic application. In certain embodiments, the anti-CEACAM6 antibody binds to an epitope which is conserved between different species.

TPP-3310 is an antibody which comprises H-CDR1 comprising the amino acid sequence of SEQ ID NO: 12, H-CDR2 comprising the amino acid sequence of SEQ ID NO: 13, H-CDR3 comprising the amino acid sequence of SEQ ID NO: 14, L-CDR1 comprising the amino acid sequence of SEQ ID NO: 16, L-CDR2 comprising the amino acid sequence of SEQ ID NO: 17, and L-CDR3 comprising the amino acid sequence of SEQ ID NO: 18.

Preferably TPP-3310 is an antibody which comprises a variable heavy chain sequence (VH) of SEQ ID NO:11 and a variable light chain sequences (VL) of SEQ ID NO:15.

Highly preferred, TPP-3310 is an antibody which comprises a heavy chain region (HC) of SEQ ID NO: 19 and a light chain region (LC) of SEQ ID NO: 20.

Component B of the Combination

Component B is an antibody or an antigen-binding portion thereof that binds specifically to a Programmed Death-1 (PD-1) receptor and inhibits PD-1 activity (“anti-PD-1 antibody”) or an antibody or an antigen-binding portion thereof that binds specifically to a Programmed Death Ligand-1 (PD-L1) and inhibits PD-L1 activity (“anti-PD-L1 antibody”).

Anti-PD-1 Antibody

In certain embodiments, the anti-PD-1 antibody or an antigen-binding portion thereof is nivolumab or has the same CDR regions as nivolumab. Nivolumab (trade name “OPDIVO”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449). In another embodiment, the anti-PD-1 antibody or fragment thereof cross competes with nivolumab.

TPP-2596 which is an anti-PD-1 human IgG4 (S228P) antibody which was cloned using the variable domains of nivolumab.

In other embodiments, the anti-PD-1 antibody or an antigen-binding portion thereof is pembrolizumab or has the same CDR regions as pembrolizumab. Pembrolizumab (trade name “KEYTRUDA”, also known as lambrolizumab, and MK-3475) is a humanized monoclonal IgG4 antibody directed against human cell surface receptor PD-1. Pembrolizumab is described, for example, in U.S. Pat. No. 8,900,587.

In other embodiments, the anti-PD-1 antibody or an antigen-binding portion thereof is MEDI0608 (formerly AMP-514) or has the same CDR regions as MEDI0608. MEDI0608 is a monoclonal antibody against the PD-1 receptor. MEDI0608 is described, for example, in U.S. Pat. No. 8,609,089, B2.

In other embodiments, the anti-PD-1 antibody or an antigen-binding portion thereof is BGB-A317 or has the same CDR regions as BGB-A317. BGB-A317 is a humanized monoclonal antibody described in U.S. Publ. No. 2015/0079109.

In certain embodiments, the anti-PD-1 antibody comprises:
(i) H-CDR1 comprising the amino acid sequence of SEQ ID NO: 2, H-CDR2 comprising the amino acid sequence of SEQ ID NO: 3, H-CDR3 comprising the amino acid sequence of SEQ ID NO: 4, L-CDR1 comprising the amino acid sequence of SEQ ID NO: 6, L-CDR2 comprising the amino acid sequence of SEQ ID NO: 7, and L-CDR3 comprising the amino acid sequence of SEQ ID NO: 8, or
(ii) H-CDR1 comprising the amino acid sequence of SEQ ID NO: 32, H-CDR2 comprising the amino acid sequence of SEQ ID NO: 33, H-CDR3 comprising the amino acid sequence of SEQ ID NO: 34, L-CDR1 comprising the amino acid sequence of SEQ ID NO: 36, L-CDR2 comprising the amino acid sequence of SEQ ID NO: 37, and L-CDR3 comprising the amino acid sequence of SEQ ID NO: 38.

In certain embodiments, the anti-PD-1 antibody comprises:
(i) a variable heavy chain sequence (VH) of SEQ ID NO:1 and a variable light chain sequences (VL) of SEQ ID NO:5, or
(ii) a variable heavy chain sequence (VH) of SEQ ID NO:31 and a variable light chain sequences (VL) of SEQ ID NO:35.

In certain embodiments, the anti-PD-1 antibody comprises:
(i) a heavy chain region (HC) of SEQ ID NO: 9 and a light chain region (LC) of SEQ ID NO: 10, or
(ii) a heavy chain region (HC) of SEQ ID NO: 39 and a light chain region (LC) of SEQ ID NO: 40.

Anti-PD-L1 Antibody

In certain embodiments, the anti-PD-L1 antibody or an antigen-binding portion thereof is atezolizumab or has the same CDR regions as atezolizumab. Atezolizumab (trade name “TECENTRIQ”) also known as MPDL3280A, RG7446) is described in U.S. Pat. No. 8,217,149.

TPP-3615 is an anti-PD-L1 hulgG2 antibody which was cloned using the variable domains of atezolizumab.

In other embodiments, the anti-PD-L1 antibody or an antigen-binding portion thereof is avelumab or has the same CDR regions as avelumab. Avelumab (trade name “BAVENCIO”) also known as MSB0010718C is described in US 2014/0341917.

In other embodiments, the anti-PD-L1 antibody or an antigen-binding portion thereof is durvalumab or has the same CDR regions as durvalumab. Durvalumab (trade name “IMFINZI”) also known as MEDI4736) is described in U.S. Pat. No. 8,779,108 or US 2014/0356353.

In other embodiments, the anti-PD-L1 antibody or an antigen-binding portion thereof is BMS-936559 or has the same CDR regions as BMS-936559. BMS-936559 (formerly 12A4 or MDX-1105) is a fully human IgG4 monoclonal antibody that targets the PD-1 ligand PD-L1 and is described in U.S. Pat. No. 7,943,743 or WO 2013/173223.

In certain embodiments, the anti-PD-L1 antibody comprises:
(iii) H-CDR1 comprising the amino acid sequence of SEQ ID NO: 42, H-CDR2 comprising the amino acid sequence of SEQ ID NO: 43, H-CDR3 comprising the amino acid sequence of SEQ ID NO: 44, L-CDR1 comprising the amino acid sequence of SEQ ID NO: 46, L-CDR2 comprising the amino acid sequence of SEQ ID NO: 47, and L-CDR3 comprising the amino acid sequence of SEQ ID NO: 48, or
(iv) H-CDR1 comprising the amino acid sequence of SEQ ID NO: 52, H-CDR2 comprising the amino acid sequence of SEQ ID NO: 53, H-CDR3 comprising the amino acid sequence of SEQ ID NO: 54, L-CDR1 comprising the amino acid sequence of SEQ ID NO: 56, L-CDR2 comprising the amino acid sequence of SEQ ID NO: 57, and L-CDR3 comprising the amino acid sequence of SEQ ID NO: 58, or
(v) H-CDR1 comprising the amino acid sequence of SEQ ID NO: 62, H-CDR2 comprising the amino acid sequence of SEQ ID NO: 63, H-CDR3 comprising the amino acid sequence of SEQ ID NO: 64, L-CDR1 comprising the amino acid sequence of SEQ ID NO: 66, L-CDR2 comprising the amino acid sequence of SEQ ID NO: 67, and L-CDR3 comprising the amino acid sequence of SEQ ID NO: 68, or
(vi) H-CDR1 comprising the amino acid sequence of SEQ ID NO: 22, H-CDR2 comprising the amino acid sequence of SEQ ID NO: 23, H-CDR3 comprising the amino acid sequence of SEQ ID NO: 24, L-CDR1 comprising the amino acid sequence of SEQ ID NO: 26, L-CDR2 comprising the amino acid sequence of SEQ ID NO: 27, and L-CDR3 comprising the amino acid sequence of SEQ ID NO: 28.

In certain embodiments, the anti-PD-L1 antibody comprises:
(iii) a variable heavy chain sequence (VH) of SEQ ID NO:41 and a variable light chain sequences (VL) of SEQ ID NO:45, or
(iv) a variable heavy chain sequence (VH) of SEQ ID NO:51 and a variable light chain sequences (VL) of SEQ ID NO:55, or
(v) a variable heavy chain sequence (VH) of SEQ ID NO:61 and a variable light chain sequences (VL) of SEQ ID NO:65, or
(vi) a variable heavy chain sequence (VH) of SEQ ID NO:21 and a variable light chain sequences (VL) of SEQ ID NO:25.

In certain embodiments, the anti-PD-L1 antibody comprises:
(iii) a heavy chain region (HC) of SEQ ID NO: 49 and a light chain region (LC) of SEQ ID NO: 50, or
(iv) a heavy chain region (HC) of SEQ ID NO: 59 and a light chain region (LC) of SEQ ID NO: 60, or
(v) a heavy chain region (HC) of SEQ ID NO: 69 and a light chain region (LC) of SEQ ID NO: 70, or
(vi) a heavy chain region (HC) of SEQ ID NO: 29 and a light chain region (LC) of SEQ ID NO: 30.

Production of Antibodies

Antibodies or antigen-binding antibody fragments which bind target molecules may be prepared by a person of ordinary skill in the art using known processes, such as, for example, chemical synthesis or recombinant expression. Binders for cancer target molecules may be acquired commercially or may be prepared by a person of ordinary skill in the art using known processes, such as, for example, chemical synthesis or recombinant expression. Further processes for preparing antibodies or antigen-binding antibody fragments are described in WO 2007/070538 (see page 22 “Antibodies”). The person skilled in the art knows how processes such as phage display libraries (e.g. Morphosys HuCAL Gold) can be compiled and used for discovering antibodies or antigen-binding antibody fragments (see WO 2007/070538, page 24 ff and AK Example 1 on page 70, AK Example 2 on page 72). Further processes for preparing antibodies that use DNA libraries from B cells are described for example on page 26 (WO 2007/070538). Processes for humanizing antibodies are described on page 30-32 of WO2007070538 and in detail in Queen, et al., Pros. Natl. Acad. Sci. USA 8610029-10033,1989 or in WO 90/0786. Furthermore, processes for recombinant expression of proteins in general and of antibodies in particular are known to the person skilled in the art (see, for example, in Berger and Kimmel (Guide to Molecular Cloning Techniques, Methods in Enzymology, Vol. 152, Academic Press, Inc.); Sambrook, et al., (Molecular Cloning A Laboratory Manual, (Second Edition, Cold Spring Harbor Laboratory Press; Cold Spring Harbor, N.Y.; 1989) Vol. 1-3); Current Protocols in Molecular Biology, (F. M. Ausabel et al. [Eds.], Current Protocols, Green Publishing Associates, Inc./John Wiley & Sons, Inc.); Harlow et al., (Monoclonal Antibodies A Laboratory Manual, Cold Spring Harbor Laboratory Press (19881, Paul [Ed.]); Fundamental Immunology, (Lippincott Williams & Wilkins (1998)); and

Harlow, et al., (Using Antibodies A Laboratory Manual, Cold Spring Harbor Laboratory Press (1998)). The person skilled in the art knows the corresponding vectors, promoters and signal peptides which are necessary for the expression of a protein/antibody. Commonplace processes are also described in WO 2007/070538 on pages 41-45. Processes for preparing an IgG1 antibody are described for example in WO 2007/070538 in Example 6 on page 74 ff. Processes which allow the determination of the internalization of an antibody after binding to its antigen are known to the skilled person and are described for example in WO 2007/070538 on page 80. The person skilled in the art is able to use the processes described in WO 2007/070538 that have been used for preparing carboanhydrase IX (Mn) antibodies in analogy for the preparation of antibodies with different target molecule specificity.

Bacterial Expression

The person skilled in the art is aware of the way in which antibodies, antigen-binding fragments thereof or variants thereof can be produced with the aid of bacterial expression.

Suitable expression vectors for bacterial expression of desired proteins are constructed by insertion of a DNA sequence which encodes the desired protein within the functional reading frame together with suitable translation initiation and translation termination signals and with a functional promoter. The vector comprises one or more phenotypically selectable markers and a replication origin in order to enable the retention of the vector and, if desired, the amplification thereof within the host. Suitable prokaryotic hosts for transformation include but are not limited to E. coli, Bacillus subtilis, Salmonella typhimurium and various species from the genus Pseudomonas, Streptomyces, and Staphylococcus. Bacterial vectors may be based, for example, on bacteriophages, plasmids, or phagemids. These vectors may contain selectable markers and a bacterial replication origin, which are derived from commercially available plasmids. Many commercially available plasmids typically contain elements of the well-known cloning vector pBR322 (ATCC 37017). In bacterial systems, a number of advantageous expression vectors can be selected on the basis of the intended use of the protein to be expressed.

After transformation of a suitable host strain and growth of the host strain to an appropriate cell density, the selected promoter is de-reprimed/induced by suitable means (for example a change in temperature or chemical induction), and the cells are cultivated for an additional period. The cells are typically harvested by centrifugation and if necessary digested in a physical manner or by chemical means, and the resulting raw extract is retained for further purification.

Mammalian Cell Expression

The person skilled in the art is aware of the way in which antibodies, antigen-binding fragments thereof or variants thereof can be produced with the aid of mammalian cell expression.

Preferred regulatory sequences for expression in mammalian cell hosts include viral elements which lead to high expression in mammalian cells, such as promoters and/or expression amplifiers derived from cytomegalovirus (CMV) (such as the CMV promoter/enhancer), simian virus 40 (SV40) (such as the SV40 promoter/enhancer), from adenovirus, (for example the adenovirus major late promoter (AdMLP)) and from polyoma. The expression of the antibodies may be constitutive or regulated (for example induced by addition or removal of small molecule inductors such as tetracycline in combination with the Tet system).

For further description of viral regulatory elements and sequences thereof, reference is made, for example, to U.S. Pat. No. 5,168,062 by Stinski, U.S. Pat. No. 4,510,245 by Bell et al. and U.S. Pat. No. 4,968,615 by Schaffner et al. The recombinant expression vectors may likewise include a replication origin and selectable markers (see, for example, U.S. Pat. Nos. 4,399,216, 4,634,665 and 5,179,017). Suitable selectable markers include genes which impart resistance to substances such as G418, puromycin, hygromycin, blasticidin, zeocin/bleomycin, or methotrexate, or selectable markers which lead to auxotrophy of a host cell, such as glutamine synthetase (Bebbington et al., Biotechnology (N Y). 1992 February; 10(2):169-75), when the vector has been introduced into the cell.

For example, the dihydrofolate reductase (DHFR) gene imparts resistance to methotrexate, the neo gene imparts resistance to G418, the bsd gene from Aspergillus terreus imparts resistance to blasticidin, puromycin N-acetyltransferase imparts resistance to puromycin, the Sh ble gene product imparts resistance to zeocin, and resistance to hygromycin is imparted by the E. coli hygromycin resistance gene (hyg or hph). Selectable markers such as DHFR or glutamine synthetase are also helpful for amplification techniques in conjunction with MTX and MSX.

The transfection of an expression vector into a host cell can be executed with the aid of standard techniques, including by electroporation, nucleofection, calcium phosphate precipitation, lipofection, polycation-based transfection such as polyethyleneimine (PEI)-based transfection and DEAE-dextran transfection.

Suitable mammalian host cells for the expression of antibodies, antigen-binding fragments thereof, or variants thereof include Chinese hamster ovary (CHO) cells such as CHO-K1, CHO-S, CHO-K1SV [including DHFR-CHO cells, described in Urlaub and Chasin, (1980) Proc. Natl. Acad. Sci. USA 77:4216-4220 and Urlaub et al., Cell. 1983 June; 33(2):405-12, used with a DHFR-selectable marker, as described in R. J. Kaufman and P. A. Sharp (1982) Mol. Biol. 159:601-621, and other knockout cells, as detailed in Fan et al., Biotechnol Bioeng. 2012 April; 109(4):1007-15), NS0 myeloma cells, COS cells, HEK293 cells, HKB11 cells, BHK21 cells, CAP cells, EB66 cells, and SP2 cells.

The expression of antibodies, antigen-binding fragments thereof, or variants thereof can also be effected in a transient or semi-stable manner in expression systems such as HEK293, HEK293T, HEK293-EBNA, HEK293E, HEK293-6E, HEK293 Freestyle, HKB11, Expi293F, 293EBNALT75, CHO Freestyle, CHO-S, CHO-K1, CHO-K1SV, CHOEBNALT85, CHOS-XE, CHO-3E7 or CAP-T cells (for example like Durocher et al., Nucleic Acids Res. 2002 Jan. 15; 30(2):E9).

In some embodiments, the expression vector is constructed in such a way that the protein to be expressed is secreted into the cell culture medium in which the host cells are growing. The antibodies, the antigen-binding fragments thereof, or the variants thereof can be obtained from the cell culture medium with the aid of protein purification methods known to those skilled in the art.

### Purification

The antibodies, the antigen-binding fragments thereof, or the variants thereof can be obtained and purified from recombinant cell cultures with the aid of well-known methods, examples of which include ammonium sulfate or ethanol precipitation, acid extraction, protein A chromatography, protein G chromatography, anion or cation exchange chromatography, phosphocellulose chromatography, hydrophobic interaction chromatography (HIC), affinity chromatography, hydroxyapatite chromatography and lectin chromatography. High-performance liquid chromatography (“HPLC”) can likewise be employed for purification. See, for example, Colligan, Current Protocols in Immunology, or Current Protocols in Protein Science, John Wiley & Sons, NY, N.Y., (1997-2001), e.g., Chapters 1, 4, 6, 8, 9, 10.

Antibodies of the present invention or antigen-binding fragments thereof, or variants thereof include naturally purified products, products from chemical synthesis methods and products which are produced with the aid of recombinant techniques in prokaryotic or eukaryotic host cells. Eukaryotic hosts include, for example, yeast cells, higher plant cells, insect cells and mammalian cells. Depending on the host cell chosen for the recombinant expression, the protein expressed may be in glycosylated or non-glycosylated form.

In a preferred embodiment, the antibody is purified (1) to an extent of more than 95% by weight, measured, for example, by the Lowry method, by UV-vis spectroscopy or by SDS capillary gel electrophoresis (for example with a Caliper LabChip GXII, GX 90 or Biorad Bioanalyzer instrument), and in more preferred embodiments more than 99% by weight, (2) to a degree suitable for determination of at least 15 residues of the N-terminal or internal amino acid sequence, or (3) to homogeneity determined by SDS-PAGE under reducing or non-reducing conditions with the aid of Coomassie blue or preferably silver staining.

Usually, an isolated antibody is obtained with the aid of at least one protein purification step.

TABLE 1
Protein sequences of preferred antibodies

| | | | | | | | | | | SEQ ID | SEQ ID |
|---|---|---|---|---|---|---|---|---|---|---|---|
| | | SEQ ID | SEQ | SEQ | SEQ | SEQ ID | SEQ | SEQ | SEQ | NO: IgG | NO: IgG |
| | Antibody | NO: VH | ID NO: | ID NO: | ID NO: | NO: VL | ID NO: | ID NO: | ID NO: | Heavy | Light |
| TPP-ID | [Name] | Protein | H-CDR1 | H-CDR2 | H-CDR3 | Protein | L-CDR1 | L-CDR2 | L-CDR3 | Chain | Chain |
| TPP-2596 | Nivolumab | 1 | 2 | 3 | 4 | 5 | 6 | 7 | 8 | 9 | 10 |
| TPP-3310 | aCECAM6 | 11 | 12 | 13 | 14 | 15 | 16 | 17 | 18 | 19 | 20 |
| TPP-3615 | Atezolizu-mab | 21 | 22 | 23 | 24 | 25 | 26 | 27 | 28 | 29 | 30 |
| | variant | | | | | | | | | | |
| TPP-9692 | Pembrolizu-mab | 31 | 32 | 33 | 34 | 35 | 36 | 37 | 38 | 39 | 40 |
| TPP-17802 | Atezolizu-mab | 41 | 42 | 43 | 44 | 45 | 46 | 47 | 48 | 49 | 50 |
| TPP-17803 | Avelumab | 51 | 52 | 53 | 54 | 55 | 56 | 57 | 58 | 59 | 60 |
| TPP-17804 | Durvalu-mab | 61 | 62 | 63 | 64 | 65 | 66 | 67 | 68 | 69 | 70 |

TABLE 2
Sequences of preferred antibodies

| ″TPP- | Antibody | | | |
|---|---|---|---|---|
| ID″ | [Name] | Region | SEQ ID | Sequence |
| TPP- | Nivolumab | VH | SEQ ID NO: 1 | QVQLVESGGGVVQPGRSLRLDC |
| 2596 | | | | KASGITFSNSGMHWVRQAPGKG |
| | | | | LEWVAVIWYDGSKRYYADSVKG |
| | | | | RFTISRDNSKNTLFLQMNSLRAE |
| | | | | DTAVYYCATNDDYWGQGTLVTV |
| | | | | SS |
| TPP- | Nivolumab | HCDR1 | SEQ ID NO: 2 | NSGMH |
| 2596 | | | | |
| TPP- | Nivolumab | HCDR2 | SEQ ID NO: 3 | VIWYDGSKRYYADSVKG |
| 2596 | | | | |
| TPP- | Nivolumab | HCDR3 | SEQ ID NO: 4 | NDDY |
| 2596 | | | | |
| TPP- | Nivolumab | VL | SEQ ID NO: 5 | EIVLTQSPATLSLSPGERATLSCR |
| 2596 | | | | ASQSVSSYLAWYQQKPGQAPRL |
| | | | | LIYDASNRATGIPARFSGSGSGTD |
| | | | | FTLTISSLEPEDFAVYYCQQSSN |
| | | | | WPRTFGQGTKVEIK |
| TPP- | Nivolumab | LCDR1 | SEQ ID NO: 6 | RASQSVSSYLA |
| 2596 | | | | |
| TPP- | Nivolumab | LCDR2 | SEQ ID NO: 7 | DASNRAT |
| 2596 | | | | |
| TPP- | Nivolumab | LCDR3 | SEQ ID NO: 8 | QQSSNWPRT |
| 2596 | | | | |
| TPP- | Nivolumab | Heavy | SEQ ID NO: 9 | QVQLVESGGGVVQPGRSLRLDC |
| 2596 | | Chain | | KASGITFSNSGMHWVRQAPGKG |
| | | | | LEWVAVIWYDGSKRYYADSVKG |
| | | | | RFTISRDNSKNTLFLQMNSLRAE |
| | | | | DTAVYYCATNDDYWGQGTLVTV |
| | | | | SSASTKGPSVFPLAPCSRSTSES |
| | | | | TAALGCLVKDYFPEPVTVSWNSG |
| | | | | ALTSGVHTFPAVLQSSGLYSLSS |
| | | | | VVTVPSSSLGTKTYTCNVDHKPS |
| | | | | NTKVDKRVESKYGPPCPPCPAPE |
| | | | | FLGGPSVFLFPPKPKDTLMISRTP |
| | | | | EVTCVVVDVSQEDPEVQFNWYV |
| | | | | DGVEVHNAKTKPREEQFNSTYRV |
| | | | | VSVLTVLHQDWLNGKEYKCKVSN |
| | | | | KGLPSSIEKTISKAKGQPREPQVY |
| | | | | TLPPSQEEMTKNQVSLTCLVKGF |
| | | | | YPSDIAVEWESNGQPENNYKTTP |
| | | | | PVLDSDGSFFLYSRLTVDKSRWQ |
| | | | | EGNVFSCSVMHEALHNHYTQKSL |
| | | | | SLSLGK |
| TPP- | Nivolumab | Light | SEQ ID | EIVLTQSPATLSLSPGERATLSCR |
| 2596 | | Chain | NO: 10 | ASQSVSSYLAWYQQKPGQAPRL |
| | | | | LIYDASNRATGIPARFSGSGSGTD |
| | | | | FTLTISSLEPEDFAVYYCQQSSN |
| | | | | WPRTFGQGTKVEIKRTVAAPSVFI |
| | | | | FPPSDEQLKSGTASVVCLLNNFY |
| | | | | PREAKVQWKVDNALQSGNSQES |
| | | | | VTEQDSKDSTYSLSSTLTLSKADY |
| | | | | EKHKVYACEVTHQGLSSPVTKSF |
| | | | | NRGEC |
| TPP- | aCECAM6 | VH | SEQ ID | QVTLRESGPALVKPTQTLTLTCTF |
| 3310 | | | NO: 11 | SGFSLSTYGIGVGWIRQPPGKAL |
| | | | | EWLAHIWWNDNKYYSTSLKTRLT |
| | | | | ISKDTSKNQVVLTMTNMDPVDTA |
| | | | | TYYCARISLPYFDYWGQGTTLTV |
| | | | | SS |
| TPP- | aCECAM6 | HCDR1 | SEQ ID | TYGIGVG |
| 3310 | | | NO: 12 | |
| TPP- | aCECAM6 | HCDR2 | SEQ ID | HIWWNDNKYYSTSLKT |
| 3310 | | | NO: 13 | |
| TPP- | aCECAM6 | HCDR3 | SEQ ID | ISLPYFDY |
| 3310 | | | NO: 14 | |
| TPP- | aCECAM6 | VL | SEQ ID | DIQLTQSPSFLSASVGDRVTITCK |
| 3310 | | | NO: 15 | ASQNVGTAVAWYQQKPGKAPKL |
| | | | | LIYSASNRYTGVPSRFSGSGSGT |
| | | | | EFTLTISSLQPEDFATYYCQQYSS |
| | | | | YPLTFGGGTKVEIK |
| TPP- | aCECAM6 | LCDR1 | SEQ ID | KASQNVGTAVA |
| 3310 | | | NO: 16 | |
| TPP- | aCECAM6 | LCDR2 | SEQ ID | SASNRYT |
| 3310 | | | NO: 17 | |
| TPP- | aCECAM6 | LCDR3 | SEQ ID | QQYSSYPLT |
| 3310 | | | NO: 18 | |
| TPP- | aCECAM6 | Heavy | SEQ ID | QVTLRESGPALVKPTQTLTLTCTF |
| 3310 | | Chain | NO: 19 | SGFSLSTYGIGVGWIRQPPGKAL |
| | | | | EWLAHIWWNDNKYYSTSLKTRLT |
| | | | | ISKDTSKNQVVLTMTNMDPVDTA |
| | | | | TYYCARISLPYFDYWGQGTTLTV |
| | | | | SSASTKGPSVFPLAPCSRSTSES |
| | | | | TAALGCLVKDYFPEPVTVSWNSG |
| | | | | ALTSGVHTFPAVLQSSGLYSLSS |
| | | | | VVTVPSSNFGTQTYTCNVDHKPS |
| | | | | NTKVDKTVERKCCVECPPCPAPP |
| | | | | VAGPSVFLFPPKPKDTLMISRTPE |
| | | | | VTCVVVDVSHEDPEVQFNWYVD |
| | | | | GVEVHNAKTKPREEQFNSTFRVV |
| | | | | SVLTVVHQDWLNGKEYKCKVSN |
| | | | | KGLPAPIEKTISKTKGQPREPQVY |
| | | | | TLPPSREEMTKNQVSLTCLVKGF |
| | | | | YPSDIAVEWESNGQPENNYKTTP |
| | | | | PMLDSDGSFFLYSKLTVDKSRWQ |
| | | | | QGNVFSCSVMHEALHNHYTQKS |
| | | | | LSLSPG |
| TPP- | aCECAM6 | Light | SEQ ID | DIQLTQSPSFLSASVGDRVTITCK |
| 3310 | | Chain | NO: 20 | ASQNVGTAVAWYQQKPGKAPKL |
| | | | | LIYSASNRYTGVPSRFSGSGSGT |
| | | | | EFTLTISSLQPEDFATYYCQQYSS |
| | | | | YPLTFGGGTKVEIKRTVAAPSVFI |
| | | | | FPPSDEQLKSGTASVVCLLNNFY |
| | | | | PREAKVQWKVDNALQSGNSQES |
| | | | | VTEQDSKDSTYSLSSTLTLSKADY |
| | | | | EKHKVYACEVTHQGLSSPVTKSF |
| | | | | NRGEC |
| TPP- | Atezolizumab | VH | SEQ ID | EVQLVESGGGLVQPGGSLRLSC |
| 3615 | var. | | NO: 21 | AASGFTFSDSWIHWVRQAPGKG |
| | | | | LEWVAWISPYGGSTYYADSVKG |
| | | | | RFTISADTSKNTAYLQMNSLRAE |
| | | | | DTAVYYCARRHWPGGFDYWGQ |
| | | | | GTLVTVSA |
| TPP- | Atezolizumab | HCDR1 | SEQ ID | DSWIH |
| 3615 | var. | | NO: 22 | |
| TPP- | Atezolizumab | HCDR2 | SEQ ID | WISPYGGSTYYADSVKG |
| 3615 | var. | | NO: 23 | |
| TPP- | Atezolizumab | HCDR3 | SEQ ID | ARRHWPGGFDY |
| 3615 | var. | | NO: 24 | |
| TPP- | Atezolizumab | VL | SEQ ID | DIQMTQSPSSLSASVGDRVTITCR |
| 3615 | var. | | NO: 25 | ASQDVSTAVAWYQQKPGKAPKL |
| | | | | LIYSASFLYSGVPSRFSGSGSGT |
| | | | | DFTLTISSLQPEDFATYYCQQYLY |
| | | | | HPATFGQGTKVEIK |
| TPP- | Atezolizumab | LCDR1 | SEQ ID | RASQDVSTAVA |
| 3615 | var. | | NO: 26 | |
| TPP- | Atezolizumab | LCDR2 | SEQ ID | SASFLYS |
| 3615 | var. | | NO: 27 | |
| TPP- | Atezolizumab | LCDR3 | SEQ ID | QQYLYHPAT |
| 3615 | var. | | NO: 28 | |
| TPP- | Atezolizumab | Heavy | SEQ ID | EVQLVESGGGLVQPGGSLRLSC |
| 3615 | var. | Chain | NO: 29 | AASGFTFSDSWIHWVRQAPGKG |
| | | | | LEWVAWISPYGGSTYYADSVKG |
| | | | | RFTISADTSKNTAYLQMNSLRAE |
| | | | | DTAVYYCARRHWPGGFDYWGQ |
| | | | | GTLVTVSASSASTKGPSVFPLAP |
| | | | | CSRSTSESTAALGCLVKDYFPEP |
| | | | | VTVSWNSGALTSGVHTFPAVLQS |
| | | | | SGLYSLSSVVTVPSSNFGTQTYT |
| | | | | CNVDHKPSNTKVDKTVERKCCVE |
| | | | | CPPCPAPPVAGPSVFLFPPKPKD |
| | | | | TLMISRTPEVTCVVVDVSHEDPE |
| | | | | VQFNWYVDGVEVHNAKTKPREE |
| | | | | QFNSTFRVVSVLTVVHQDWLNG |
| | | | | KEYKCKVSNKGLPAPIEKTISKTK |
| | | | | GQPREPQVYTLPPSREEMTKNQ |
| | | | | VSLTCLVKGFYPSDIAVEWESNG |
| | | | | QPENNYKTTPPMLDSDGSFFLYS |
| | | | | KLTVDKSRWQQGNVFSCSVMHE |
| | | | | ALHNHYTQKSLSLSPG |
| TPP- | Atezolizumab | Light | SEQ ID | DIQMTQSPSSLSASVGDRVTITCR |
| 3615 | var. | Chain | NO: 30 | ASQDVSTAVAWYQQKPGKAPKL |
| | | | | LIYSASFLYSGVPSRFSGSGSGT |
| | | | | DFTLTISSLQPEDFATYYCQQYLY |
| | | | | HPATFGQGTKVEIKRTVAAPSVFI |
| | | | | FPPSDEQLKSGTASVVCLLNNFY |
| | | | | PREAKVQWKVDNALQSGNSQES |
| | | | | VTEQDSKDSTYSLSSTLTLSKADY |
| | | | | EKHKVYACEVTHQGLSSPVTKSF |
| | | | | NRGEC |
| TPP- | Pembrolizumab | VH | SEQ ID | QVQLVQSGVEVKKPGASVKVSC |
| 9692 | | | NO: 31 | KASGYTFTNYYMYWVRQAPGQG |
| | | | | LEWMGGINPSNGGTNFNEKFKN |
| | | | | RVTLTTDSSTTTAYMELKSLQFD |
| | | | | DTAVYYCARRDYRFDMGFDYWG |
| | | | | QGTTVTVSS |
| TPP- | Pembrolizumab | HCDR1 | SEQ ID | NYYMY |
| 9692 | | | NO: 32 | |
| TPP- | Pembrolizumab | HCDR2 | SEQ ID | GINPSNGGTNFNEKFKN |
| 9692 | | | NO: 33 | |
| TPP- | Pembrolizumab | HCDR3 | SEQ ID | RDYRFDMGFDY |
| 9692 | | | NO: 34 | |
| TPP- | Pembrolizumab | VL | SEQ ID | EIVLTQSPATLSLSPGERATLSCR |
| 9692 | | | NO: 35 | ASKGVSTSGYSYLHVVYQQKPGQ |
| | | | | APRLLIYLASYLESGVPARFSGSG |
| | | | | SGTDFTLTISSLEPEDFAVYYCQH |
| | | | | SRDLPLTFGGGTKVEIK |
| TPP- | Pembrolizumab | LCDR1 | SEQ ID | RASKGVSTSGYSYLH |
| 9692 | | | NO: 36 | |
| TPP- | Pembrolizumab | LCDR2 | SEQ ID | LASYLES |
| 9692 | | | NO: 37 | |
| TPP- | Pembrolizumab | LCDR3 | SEQ ID | QHSRDLPLT |
| 9692 | | | NO: 38 | |
| TPP- | Pembrolizumab | Heavy | SEQ ID | QVQLVQSGVEVKKPGASVKVSC |
| 9692 | | Chain | NO: 39 | KASGYTFTNYYMYWVRQAPGQG |
| | | | | LEWMGGINPSNGGTNFNEKFKN |
| | | | | RVTLTTDSSTTTAYMELKSLQFD |
| | | | | DTAVYYCARRDYRFDMGFDYWG |
| | | | | QGTTVTVSSASTKGPSVFPLAPC |
| | | | | SRSTSESTAALGCLVKDYFPEPV |
| | | | | TVSWNSGALTSGVHTFPAVLQSS |
| | | | | GLYSLSSVVTVPSSSLGTKTYTC |
| | | | | NVDHKPSNTKVDKRVESKYGPP |
| | | | | CPPCPAPEFLGGPSVFLFPPKPK |
| | | | | DTLMISRTPEVTCVVVDVSQEDP |
| | | | | EVQFNWYVDGVEVHNAKTKPRE |
| | | | | EQFNSTYRVVSVLTVLHQDWLNG |
| | | | | KEYKCKVSNKGLPSSIEKTISKAK |
| | | | | GQPREPQVYTLPPSQEEMTKNQ |
| | | | | VSLTCLVKGFYPSDIAVEWESNG |
| | | | | QPENNYKTTPPVLDSDGSFFLYS |
| | | | | RLTVDKSRWQEGNVFSCSVMHE |
| | | | | ALHNHYTQKSLSLSLGK |
| TPP- | Pembrolizumab | Light | SEQ ID | EIVLTQSPATLSLSPGERATLSCR |
| 9692 | | Chain | NO: 40 | ASKGVSTSGYSYLHWYQQKPGQ |
| | | | | APRLLIYLASYLESGVPARFSGSG |
| | | | | SGTDFTLTISSLEPEDFAVYYCQH |
| | | | | SRDLPLTFGGGTKVEIKRTVAAPS |
| | | | | VFIFPPSDEQLKSGTASVVCLLNN |
| | | | | FYPREAKVQWKVDNALQSGNSQ |
| | | | | ESVTEQDSKDSTYSLSSTLTLSKA |
| | | | | DYEKHKVYACEVTHQGLSSPVTK |
| | | | | SFNRGEC |
| TPP- | Atezolizumab | VH | SEQ ID | EVQLVESGGGLVQPGGSLRLSC |
| 17802 | | | NO: 41 | AASGFTFSDSWIHWVRQAPGKG |
| | | | | LEWVAWISPYGGSTYYADSVKG |
| | | | | RFTISADTSKNTAYLQMNSLRAE |
| | | | | DTAVYYCARRHWPGGFDYWGQ |
| | | | | GTLVTVSS |
| TPP- | Atezolizumab | HCDR1 | SEQ ID | DSWIH |
| 17802 | | | NO: 42 | |
| TPP- | Atezolizumab | HCDR2 | SEQ ID | WISPYGGSTYYADSVKG |
| 17802 | | | NO: 43 | |
| TPP- | Atezolizumab | HCDR3 | SEQ ID | RHWPGGFDY |
| 17802 | | | NO: 44 | |
| TPP- | Atezolizumab | VL | SEQ ID | DIQMTQSPSSLSASVGDRVTITCR |
| 17802 | | | NO: 45 | ASQDVSTAVAWYQQKPGKAPKL |
| | | | | LIYSASFLYSGVPSRFSGSGSGT |
| | | | | DFTLTISSLQPEDFATYYCQQYLY |
| | | | | HPATFGQGTKVEIK |
| TPP- | Atezolizumab | LCDR1 | SEQ ID | RASQDVSTAVA |
| 17802 | | | NO: 46 | |
| TPP- | Atezolizumab | LCDR2 | SEQ ID | SASFLYS |
| 17802 | | | NO: 47 | |
| TPP- | Atezolizumab | LCDR3 | SEQ ID | QQYLYHPAT |
| 17802 | | | NO: 48 | |
| TPP- | Atezolizumab | Heavy | SEQ ID | EVQLVESGGGLVQPGGSLRLSC |
| 17802 | | Chain | NO: 49 | AASGFTFSDSWIHWVRQAPGKG |
| | | | | LEWVAWISPYGGSTYYADSVKG |
| | | | | RFTISADTSKNTAYLQMNSLRAE |
| | | | | DTAVYYCARRHWPGGFDYWGQ |
| | | | | GTLVTVSSASTKGPSVFPLAPSS |
| | | | | KSTSGGTAALGCLVKDYFPEPVT |
| | | | | VSWNSGALTSGVHTFPAVLQSS |
| | | | | GLYSLSSVVTVPSSSLGTQTYICN |
| | | | | VNHKPSNTKVDKKVEPKSCDKTH |
| | | | | TCPPCPAPELLGGPSVFLFPPKP |
| | | | | KDTLMISRTPEVTCVVVDVSHED |
| | | | | PEVKFNWYVDGVEVHNAKTKPR |
| | | | | EEQYASTYRVVSVLTVLHQDWLN |
| | | | | GKEYKCKVSNKALPAPIEKTISKA |
| | | | | KGQPREPQVYTLPPSREEMTKN |
| | | | | QVSLTCLVKGFYPSDIAVEWESN |
| | | | | GQPENNYKTTPPVLDSDGSFFLY |
| | | | | SKLTVDKSRWQQGNVFSCSVMH |
| | | | | EALHNHYTQKSLSLSPGK |
| TPP- | Atezolizumab | Light | SEQ ID | DIQMTQSPSSLSASVGDRVTITCR |
| 17802 | | Chain | NO: 50 | ASQDVSTAVAWYQQKPGKAPKL |
| | | | | LIYSASFLYSGVPSRFSGSGSGT |
| | | | | DFTLTISSLQPEDFATYYCQQYLY |
| | | | | HPATFGQGTKVEIKRTVAAPSVFI |
| | | | | FPPSDEQLKSGTASVVCLLNNFY |
| | | | | PREAKVQWKVDNALQSGNSQES |
| | | | | VTEQDSKDSTYSLSSTLTLSKADY |
| | | | | EKHKVYACEVTHQGLSSPVTKSF |
| | | | | NRGEC |
| TPP- | Avelumab | VH | SEQ ID | EVQLLESGGGLVQPGGSLRLSCA |
| 17803 | | | NO: 51 | ASGFTFSSYIMMWVRQAPGKGL |
| | | | | EWVSSIYPSGGITFYADTVKGRFT |
| | | | | ISRDNSKNTLYLQMNSLRAEDTA |
| | | | | VYYCARIKLGTVTTVDYWGQGTL |
| | | | | VTVSS |
| TPP- | Avelumab | HCDR1 | SEQ ID | SYIMM |
| 17803 | | | NO: 52 | |
| TPP- | Avelumab | HCDR2 | SEQ ID | SIYPSGGITFYADTVKG |
| 17803 | | | NO: 53 | |
| TPP- | Avelumab | HCDR3 | SEQ ID | IKLGTVTTVDY |
| 17803 | | | NO: 54 | |
| TPP- | Avelumab | VL | SEQ ID | QSALTQPASVSGSPGQSITISCTG |
| 17803 | | | NO: 55 | TSSDVGGYNYVSWYQQHPGKAP |
| | | | | KLMIYDVSNRPSGVSNRFSGSKS |
| | | | | GNTASLTISGLQAEDEADYYCSS |
| | | | | YTSSSTRVFGTGTKVTVL |
| TPP- | Avelumab | LCDR1 | SEQ ID | TGTSSDVGGYNYVS |
| 17803 | | | NO: 56 | |
| TPP- | Avelumab | LCDR2 | SEQ ID | DVSNRPS |
| 17803 | | | NO: 57 | |
| TPP- | Avelumab | LCDR3 | SEQ ID | SSYTSSSTRV |
| 17803 | | | NO: 58 | |
| TPP- | Avelumab | Heavy | SEQ ID | EVQLLESGGGLVQPGGSLRLSCA |
| 17803 | | Chain | NO: 59 | ASGFTFSSYIMMWVRQAPGKGL |
| | | | | EWVSSIYPSGGITFYADTVKGRFT |
| | | | | ISRDNSKNTLYLQMNSLRAEDTA |
| | | | | VYYCARIKLGTVTTVDYWGQGTL |
| | | | | VTVSSASTKGPSVFPLAPSSKST |
| | | | | SGGTAALGCLVKDYFPEPVTVSW |
| | | | | NSGALTSGVHTFPAVLQSSGLYS |
| | | | | LSSVVTVPSSSLGTQTYICNVNHK |
| | | | | PSNTKVDKKVEPKSCDKTHTCPP |
| | | | | CPAPELLGGPSVFLFPPKPKDTL |
| | | | | MISRTPEVTCVVVDVSHEDPEVK |
| | | | | FNWYVDGVEVHNAKTKPREEQY |
| | | | | NSTYRVVSVLTVLHQDWLNGKEY |
| | | | | KCKVSNKALPAPIEKTISKAKGQP |
| | | | | REPQVYTLPPSRDELTKNQVSLT |
| | | | | CLVKGFYPSDIAVEWESNGQPEN |
| | | | | NYKTTPPVLDSDGSFFLYSKLTVD |
| | | | | KSRWQQGNVFSCSVMHEALHNH |
| | | | | YTQKSLSLSPGK |
| TPP- | Avelumab | Light | SEQ ID | QSALTQPASVSGSPGQSITISCTG |
| 17803 | | Chain | NO: 60 | TSSDVGGYNYVSWYQQHPGKAP |
| | | | | KLMIYDVSNRPSGVSNRFSGSKS |
| | | | | GNTASLTISGLQAEDEADYYCSS |
| | | | | YTSSSTRVFGTGTKVTVLGQPKA |
| | | | | NPTVTLFPPSSEELQANKATLVCL |
| | | | | ISDFYPGAVTVAWKADGSPVKAG |
| | | | | VETTKPSKQSNNKYAASSYLSLT |
| | | | | PEQWKSHRSYSCQVTHEGSTVE |
| | | | | KTVAPTECS |
| TPP- | Durvalumab | VH | SEQ ID | EVQLVESGGGLVQPGGSLRLSC |
| 17804 | | | NO: 61 | AASGFTFSRYWMSWVRQAPGK |
| | | | | GLEWVANIKQDGSEKYYVDSVKG |
| | | | | RFTISRDNAKNSLYLQMNSLRAE |
| | | | | DTAVYYCAREGGWFGELAFDYW |
| | | | | GQGTLVTVSS |
| TPP- | Durvalumab | HCDR1 | SEQ ID | RYWMS |
| 17804 | | | NO: 62 | |
| TPP- | Durvalumab | HCDR2 | SEQ ID | NIKQDGSEKYYVDSVKG |
| 17804 | | | NO: 63 | |
| TPP- | Durvalumab | HCDR3 | SEQ ID | EGGWFGELAFDY |
| 17804 | | | NO: 64 | |
| TPP- | Durvalumab | VL | SEQ ID | EIVLTQSPGTLSLSPGERATLSCR |
| 17804 | | | NO: 65 | ASQRVSSSYLAWYQQKPGQAPR |
| | | | | LLIYDASSRATGIPDRFSGSGSGT |
| | | | | DFTLTISRLEPEDFAVYYCQQYGS |
| | | | | LPWTFGQGTKVEIK |
| TPP- | Durvalumab | LCDR1 | SEQ ID | RASQRVSSSYLA |
| 17804 | | | NO: 66 | |
| TPP- | Durvalumab | LCDR2 | SEQ ID | DASSRAT |
| 17804 | | | NO: 67 | |
| TPP- | Durvalumab | LCDR3 | SEQ ID | QQYGSLPWT |
| 17804 | | | NO: 68 | |
| TPP- | Durvalumab | Heavy | SEQ ID | EVQLVESGGGLVQPGGSLRLSC |
| 17804 | | Chain | NO: 69 | AASGFTFSRYWMSWVRQAPGK |
| | | | | GLEWVANIKQDGSEKYYVDSVKG |
| | | | | RFTISRDNAKNSLYLQMNSLRAE |
| | | | | DTAVYYCAREGGWFGELAFDYW |
| | | | | GQGTLVTVSSASTKGPSVFPLAP |
| | | | | SSKSTSGGTAALGCLVKDYFPEP |
| | | | | VTVSWNSGALTSGVHTFPAVLQS |
| | | | | SGLYSLSSVVTVPSSSLGTQTYIC |
| | | | | NVNHKPSNTKVDKRVEPKSCDKT |
| | | | | HTCPPCPAPEFEGGPSVFLFPPK |
| | | | | PKDTLMISRTPEVTCVVVDVSHE |
| | | | | DPEVKFNWYVDGVEVHNAKTKP |
| | | | | REEQYNSTYRVVSVLTVLHQDWL |
| | | | | NGKEYKCKVSNKALPASIEKTISK |
| | | | | AKGQPREPQVYTLPPSREEMTK |
| | | | | NQVSLTCLVKGFYPSDIAVEWES |
| | | | | NGQPENNYKTTPPVLDSDGSFFL |
| | | | | YSKLTVDKSRWQQGNVFSCSVM |
| | | | | HEALHNHYTQKSLSLSPGK |
| TPP- | Durvalumab | Light | SEQ ID | EIVLTQSPGTLSLSPGERATLSCR |
| 17804 | | Chain | NO: 70 | ASQRVSSSYLAWYQQKPGQAPR |
| | | | | LLIYDASSRATGIPDRFSGSGSGT |
| | | | | DFTLTISRLEPEDFAVYYCQQYGS |
| | | | | LPWTFGQGTKVEIKRTVAAPSVFI |
| | | | | FPPSDEQLKSGTASVVCLLNNFY |
| | | | | PREAKVQWKVDNALQSGNSQES |
| | | | | VTEQDSKDSTYSLSSTLTLSKADY |
| | | | | EKHKVYACEVTHQGLSSPVTKSF |
| | | | | NRGEC |

Method of Treating Cancer

Within the context of the present invention, the term “cancer” includes, but is not limited to, cancers of the breast, lung, brain, reproductive organs, digestive tract, urinary tract, liver, eye, skin, head and neck, thyroid, parathyroid and their distant metastases. Those disorders also include multiple myeloma, lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to lung cancer, particularly small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, melanoma, particularly malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

The present invention relates to a method for using the combinations of the present invention, in the treatment or prophylaxis of a cancer, particularly (but not limited to) colorectal cancer, lung cancer, pancreatic cancer, breast cancer, prostate cancer, bladder cancer, gastric cancer, head and neck cancer, liver cancer, brain cancer, melanoma, endometrial cancer, lymphoma, leukemia, etc. Combinations can be utilized to inhibit, block, reduce, decrease, etc., cell proliferation and/or cell division, and/or induce apoptosis, in the treatment or prophylaxis of cancer, in particular (but not limited to) colorectal cancer, lung cancer, breast cancer, prostate cancer, bladder cancer, gastric cancer, head and neck cancer, liver cancer, brain cancer, melanoma, endometrial cancer, lymphoma, leukemia, etc. This method comprises administering to a mammal in need thereof, including a human, an amount of a combination of this invention, or a pharmaceutically acceptable salt, isomer, polymorph, metabolite, hydrate, solvate or ester thereof; etc. which is effective for the treatment or prophylaxis of cancer, in particular (but not limited to) colorectal cancer, lung cancer, pancreatic cancer, breast cancer, prostate cancer, bladder cancer, gastric cancer, head and neck cancer, liver cancer, brain cancer, melanoma, endometrial cancer, lymphoma, leukemia, etc.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

In preferred embodiments, the cancer is lung cancer, in particular non-small cell lung cancer (NSCLC), ovarian cancer, mesothelioma, pancreatic cancer, or gastric cancer, colorectal cancer, head and neck cancer, bladder cancer, bile duct cancer, breast cancer, cervical cancer, esophageal cancer.

Dose and Administration

Based upon standard laboratory techniques known to evaluate compounds useful for the treatment or prophylaxis of cancer, in particular (but not limited to) colorectal cancer, lung cancer, pancreatic cancer, breast cancer, prostate cancer, bladder cancer, gastric cancer, head and neck cancer, liver cancer, brain cancer, melanoma, endometrial cancer, lymphoma, leukemia, etc., by standard toxicity tests and by standard pharmacological assays for the determination of treatment of the conditions identified above in mammals, and by comparison of these results with the results of known medicaments that are used to treat these conditions, the effective dosage of the combinations of this invention can readily be determined for treatment of the indication. The amount of the active ingredient to be administered in the treatment of the condition can vary widely according to such considerations as the particular combination and dosage unit employed, the mode of administration, the period of treatment, the age, weight and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 30 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 2,500 mg of active ingredient, and can be administered one or more times per day or less than once a day. The average dosage for administration by injection, including intravenous, intramuscular, subcutaneous and parenteral injections, and use of infusion techniques will preferably be from 0.01 to 200 mg/kg of total body weight.

Of course the specific initial and continuing dosage regimen for each patient will vary according to the nature and severity of the condition as determined by the attending diagnostician, the activity of the specific combination employed, the age, weight and general condition of the patient, time of administration, route of administration, rate of excretion of the drug, drug combinations, and the like. The desired mode of treatment and number of doses of a combination of the present invention or a pharmaceutically acceptable salt or ester or composition thereof can be ascertained by those skilled in the art using conventional treatment tests.

Therapies Using Combinations of Component a as Described Supra, Component B as Described Supra, and Component C: One or More Further Pharmaceutical Agents.

The combinations of component A and component B of this invention can be administered as the sole pharmaceutical agent or in combination with one or more further pharmaceutical agents where the resulting combination of components A, B and C causes no unacceptable adverse effects. For example, the combinations of components A and B of this invention can be combined with component C, i.e. one or more further pharmaceutical agents, such as known anti-angiogenesis, anti-hyper-proliferative, anti-inflammatory, analgesic, immunoregulatory, diuretic, anti-arrhytmic, anti-hypercholsterolemia, anti-dyslipidemia, anti-diabetic or antiviral agents, and the like, as well as with admixtures and combinations thereof.

Component C, can be one or more pharmaceutical agents such as 131i-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab. docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, roniciclib, rucaparib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Generally, the use of component C in combination with a combination of components A and B of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

The following examples describe the feasibility of the present invention, but not restricting the invention to these Examples only.

### EXAMPLES

Effect of Combination Treatment of TPP-3310 an Antibody Against Human CEACAM6 with Antibodies Directed Against PD-L1 or PD-1 on Activation of PD-1 Positive Virus-Peptide Specific T Cells

Since CEACAM6 is not expressed in the rodent (no rodent orthologue) in vivo efficacy studies are not possible and no preclinical in vivo combination studies can be performed to evaluate the therapeutic potential of drug combinations.

Alternatively an in vitro cell assay system was established to test combinations of antibodies against CEACAM6 and PD-1 or PD-L1 for their in vitro efficacy and therapeutic potential.

In this cell assay system PD-1 positive FluM1 virus-peptide specific T cells were used as effector T cells. They were co-cultured with PD-L1 and CEACAM6 positive and FLuM1 peptide loaded cancer cells HCC2935 in the presence of checkpoint inhibitory antibody against CEACAM6, PD-1 or PD-L1 either as single agents or combinations thereof for 24h-48h. Induction of proinflammatory cytokines (IFNg) is measured as readout of efficacy.

Antibodies

Antibodies used were TPP-3310 (anti-CEACAM6) which is an hulgG2 antibody against the immune checkpoint molecule CEACAM6 which is overexpressed on cancer cells and myeloid cells, TPP-3615 which is an anti-PD-L1 hulgG2 antibody and which was cloned using the variable domains of Atezolizumab and TPP-2596 which is an anti-PD-1 human IgG4 (S228P) antibody which was cloned using the variable domains of Nivolumab. TPP-1238 (hulgG2) and TPP1240 (hulgG4) have been used as isotype control antibodies.

Cell Lines and Culture

HCC2935 cancer cells (ATCC-CRL-2869, lung adenocarcinoma) were cultured in RPMI-1640, 10% FCS, 5% CO₂. CEACAM6 and PD-L1 expression was confirmed by FACS analysis. For co-culture assays with virus-peptide specific T cells, the cancer cells were pulsed with a viral FluM1 peptide at 0.2 μg/ml or as indicated.

Generation and Cell Culture of FluM1 Virus-Peptide Specific T Cells

PD-1 expressing virus (influenza)-peptide specific T cells were generated from naïve PBMCs from HLA-A*0201+healthy donors which were obtained by Ficoll density centrifugation of buffy coats (Deutsches Rotes Kreuz, Mannheim). CD8+ T cells were enriched with MACS negative selection kit (Miltenyi, 130-096-495) according to the manufacturer's protocol. CD8 negative cells were irradiated (35 Gy) and pulsed with 1 μg/ml of the influenza HLA-A*0201 epitope GILGFVFTL (ProImmune) in X-Vivo-20 medium (Chemically Defined, Serum-free Hematopoietic Cell Medium, Lonza, #BE04-448Q) at 37° C. for 1.5 h and washed thereafter. The cells were re-stimulated with irradiated T2 cells and pulsed with 1 μg/ml of their associated GILGFVFTL peptide on day 7. On day 14, aliquots were frozen. The samples were thawed and washed immediately before they were used in functional assays. The suitability of the virus-peptide specific T cells was confirmed with tetramer (F391-4A-E, Prolmmune) staining and FACS analysis before the co-culture experiments on day 14.

In Vitro Assay: Analysis of Combined Antibody Efficacy in Co-Culture of T Cells and Cancer Cells

For the co-culture, cancer cells were detached non-enzymatically with PBS-EDTA for 5-15 min, centrifuged at 1,400 rpm for 5 min, washed and counted. Cancer cells were diluted in X-Vivo-20 (Lonza, #BE04-448Q) at 1×10⁵ cells/ml, pre-treated with TPP-3310, aPD-L1 and/or isotype control antibodies on ice for 10 min. After incubation, 10,000 target cancer cells were seeded in triplicates to 96-well ELISA U-plates.

Virus-peptide specific T cells were harvested, washed with X-Vivo-20, diluted in X-Vivo-20 at 2×10⁵ cells/ml and pre-treated with anti-PD-1 or isotype control antibodies on ice for 10 min. All antibodies were applied at a final concentration of 30 μg/ml. For the combination treatments, TPP-3310 was applied approximately at its half-maximal effective concentration (EC50) of 1 μg/ml to ensure the effects of other antibodies on the activation of T cells. The pre-treated T cells were seeded at 20,000 cells/well onto the target cancer cells.

The co-culture of cancer cells and effector T cells with the antibodies was incubated at 37° C., 5% CO₂ for approximately 20 h.

Then supernatants were collected and by centrifuging the co-culture plates at 1,400 rpm for 3 min. The IFN-γ levels in supernatants were measured by ELISA (Human IFN-γ-ELISA Set, BD, #555142) according to the manufacturer's instructions. Optical density of ELISA plates was measured with a Tecan Infinite M200 plate reader.

Data were statistically analyzed with paired or unpaired, two-tailed Student's t-test, using Microsoft Excel 2010 and GraphPad Prism 6. The results with p<0.05 were considered significant. Cytokine concentrations were calculated by standard curves. Factors or ratios were calculated by dividing values of TPP-3310 or given combinations by values of the respective isotype controls.

Result

In pre-experiments FLuM1 peptide loaded HCC2935 cancer cells were co-incubated with FluM1 virus-peptide specific T cells. Only in the presence of the cognate virus peptide IFN-γ secretion of the T cells was increased. This increase was dose dependent. IFN-γ secretion (p<0.05 to 0.0001) from the co-culture was further enhanced in the presence of the anti-CEACAM6 antibody TPP-3310, the anti-PD-L1 antibody TPP-3615 or in the presence of the anti-PD-1 antibody TPP-2596. All given as single agents. These data confirmed that the newly established cell assay system consisting of PD-1 positive FluM1 virus-peptide specific T cells and peptide loaded HCC2935 cancer cells is suitable for testing the efficacy of anti-CEACAM6, anti-PD-1 and anti-PD-L1 antibodies in benchmarking and combination experiments.

TABLE 3
Peptide-specificity of virus-peptide specific T cell activation measured
by IFNg secretion in co-culture experiments with virus-peptide loaded
HCC2935 cancer cells with or without immune checkpoint blocking
antibodies against CEACAM6, PD-1 or PD-L1.

| | IFNg [pg/ml] | | |
|---|---|---|---|
| | | | Standard |
| | | Standard | error of |
| Sample | Mean | deviation | mean |
| TC only | 3.5 | 0.5 | 0.3 |
| TC + HCC no peptide | 0.1 | 0.1 | 0.06 |
| TC + HCC/1 μg/ml FLU pep | 791.0 | 29.7 | 17.1 |
| TC + HCC/1 μg/ml FLU | 787.3 | 18.8 | 10.9 |
| pep + 30 μg/ml TPP-1238 | | | |
| TC + HCC/1 μg/ml FLU | 1143.7 | 104.5 | 60.3 |
| pep + 30 μg/ml aCEACAM6 | | | |
| (TPP-3310) | | | |
| TC + HCC/1 μg/ml FLU | 887.7 | 35.5 | 20.5 |
| pep + 30 μg/ml aPD-L1 | | | |
| (TPP-3615) | | | |
| TC + HCC/1 μg/ml FLU | 811.2 | 55.5 | 32.0 |
| pep + 30 μg/ml TPP-1240 | | | |
| TC + HCC/1 μg/ml FLU | 972.1 | 76.2 | 44.0 |
| pep + 30 μg/ml | | | |
| aPD-1 (TPP-2596) | | | |
| TC + HCC/10 μg/ml FLU peptide | 818.8 | 3.5 | 2.00 |
| TC + HCC/1 μg/ml FLU peptide | 915.0 | 29.2 | 16.9 |
| TC + HCC/0.1 μg/ml FLU peptide | 478.1 | 32.9 | 19.00 |
| TC + HCC/0.01 μg/ml FLU peptide | 56.6 | 52.5 | 30.3 |
| TC + HCC/0.001 μg/ml FLU peptide | 5.5 | 0.9 | 0.5 |
| TC + HCC 0.0001 μg/ml FLU peptide | 2.9 | 0.5 | 0.3 |

Description table: : Virus-peptide specific T cells (TC) were stimulated with HCC2935 lung cancer cells (HCC) pulsed with serial dilutions of the viral peptide. Antibodies were added at 30 μg/ml. Concentrations of secreted IFN-γ were determined by ELISA. Data are absolute amount of IFN-γ in pg/ml. TPP-3310, aCEACAM6; TPP-3615, aPD-L1; TPP-2596, aPD1; TPP-1238, isotype control for TPP-3310and TPP3615, aPD-1; TPP-1240, isotype control for TPP-2596.

The effect of the combination of the anti-CEACAM6 antibody TPP-3310 with antibodies directed to PD-L1 was determined overall in 7 independent co-culture experiments (n=7). In the presence of the antibodies we consistently saw an increase of IFNg secretion (absolute mean values) when given as single agents or in combination. In the presence of the PD-L1 antibody total IFNg was increased by 39.6 pg/ml, in the presence of the anti-CEACAM6 antibody TPP-3310 by 196.6 pg/ml and when given in combination by 279.9 pg/ml. This result shows that IFNg secretion is further enhanced upon combination of the PD-L1 antibody with the CEACAM6 antibody and that the effect on IFNg secretion is more than additive.

TABLE 4
Total IFNg secretion in co-culture experiments (n = 7) of FluM1 virus-peptide
specific T cells and FluM1 peptide loaded HCC2935 cells in the presence of
anti-CEACAM6 and anti-PD-L1 antibody as single agents or in combination

| | IFNg [pg/ml] | | | | | | | | | |
|---|---|---|---|---|---|---|---|---|---|---|
| | | | | | | | | | | Standard |
| | Experiment | | | | | | | | Standard | error of |
| Antibodies | 1 | 2 | 3 | 4 | 5 | 6 | 7 | Mean | deviation | mean |
| aPD-L1 | 70.7 | 42.0 | 41.9 | 26.3 | 46.8 | 76.1 | −26.3 | 39.6 | 33.9 | 12.8 |
| (TPP-3615) | | | | | | | | | | |
| aCEACAM6 | 192.4 | 39.3 | 76.5 | 232.5 | 162.3 | 205.8 | 467.5 | 196.6 | 138.5 | 52.4 |
| (TPP-3310) | | | | | | | | | | |
| aPD-L1 + | 363.8 | 156.0 | 121.9 | 416.7 | 305.5 | 343.0 | 252.5 | 279.9 | 52.4 | 41.3 |
| aCEACAM6 | | | | | | | | | | |
| (TPP-3615 + | | | | | | | | | | |
| TPP-3310) | | | | | | | | | | |

Description table: the HCC2935 lung cancer cells (HCC) were pulsed with the FluM1 peptide at 0.2 μg/ml to stimulate the virus-peptide specific T cells (TC) in the co-culture. Antibodies were applied at 30 μg/ml. For the combination treatments (n = 7), TPP-3310 was added at 1 μg/ml. Concentrations of secreted IFN-γ were determined by ELISA and data are isotype corrected
values and are given as pg/ml. TPP-3310, aCEACAM6; TPP-3615, aPD-L1; T-Test of mean values, p-value (<0.05): aPD-L1 vs CEACAM6, p = 0.0439; aPD-L1 vs Combination, p = 0.001; aCEACAM6 vs Combination, p = 0.16

In another study we determined the effect of the combination of the anti-CEACAM6 antibody TPP-3310 with antibodies directed against PD-1 in 7 independent co-culture experiments overall (n=7). In the presence of the antibodies we consistently saw an increase of IFNg secretion (absolute mean values) when given as single agents or in combination.

In the presence of the PD-1 antibody mean total IFNg was increased by 76.1 pg/ml, in the presence of the anti-CEACAM6 antibody TPP-3310 by 166.8 pg/ml and when given in combination by 317.9 pg/ml. This result shows that IFNg secretion is further enhanced upon combination of the PD-1 antibody with the CEACMA6 antibody and that the effect on IFNg secretion is more than additive.

TABLE 5
Total IFNg secretion in co-culture experiments (n = 7) of FluM1 virus-peptide
specific T cells and FluM1 peptide loaded HCC2935 cells in the presence of
anti-CEACAM6 and anti-PD-1 antibody as single agents or in combination

| | | | | | | | | | | Standard |
|---|---|---|---|---|---|---|---|---|---|---|
| | Experiment | | | | | | | | Standard | error of |
| Antibodies | 1 | 2 | 3 | 4 | 5 | 6 | 7 | Mean | deviation | mean |
| aPD-1 | 57.9 | 50.2 | 29.9 | 29.3 | 40.5 | 95.5 | 229.1 | 76.1 | 71.2 | 26.9 |
| (TPP-2596) | | | | | | | | | | |
| aCEACAM6 | 238.7 | 94.8 | 44.3 | 354.6 | 173.2 | 136.1 | 125.9 | 166.8 | 102.7 | 38.8 |
| (TPP-3310) | | | | | | | | | | |
| aPD-1 + | 388.7 | 195.2 | 88.6 | 495.8 | 343.4 | 326.6 | 386.9 | 317.9 | 135.3 | 51.1 |
| aCEACAM6 | | | | | | | | | | |
| (TPP-2596 + | | | | | | | | | | |
| TPP-3310) | | | | | | | | | | |

Description table: the HCC2935 lung cancer cells (HCC) were pulsed with the FluM1 peptide at 0.2 μg/ml to stimulate the virus-peptide specific T cells (TC) in the co-culture. Antibodies were applied at 30 μg/ml. For the combination treatments (n = 7), TPP-3310 was added at 1 μg/ml. Concentrations of secreted IFN-γ were determined by ELISA and data are isotype corrected
values and are given as pg/ml. TPP-3310, aCEACAM6; TPP-2596, aPD-1. T-Test of mean values, p-value (<0.05): aPD-1 vs CEACAM6, p = 0.13; aPD-L1 vs Combination, p = 0.0034; aCEACAM6 vs Combination, p = 0.0011

1. A method of treating cancer comprising administering anti-CEACAM6 antibody before, after or with an effective amount of an anti-PD-1 antibody or an anti-PD-L1 antibody in the treatment of cancer, wherein the anti-CEACAM6 antibody comprises H-CDR1 comprising the amino acid sequence of SEQ ID NO: 12, H-CDR2 comprising the amino acid sequence of SEQ ID NO: 13, H-CDR3 comprising the amino acid sequence of SEQ ID NO: 14, L-CDR1 comprising the amino acid sequence of SEQ ID NO: 16, L-CDR2 comprising the amino acid sequence of SEQ ID NO: 17, and L-CDR3 comprising the amino acid sequence of SEQ ID NO: 18.

2. The method of claim 1, wherein the anti-CEACAM6 antibody comprises a variable heavy chain sequence of SEQ ID NO:11 and a variable light chain sequence of SEQ ID NO:15.

3. The method of claim 1, wherein the anti-CEACAM6 antibody comprises a heavy chain region of SEQ ID NO: 19 and a light chain region of SEQ ID NO: 20.

4. The method of claim 1, wherein the anti-PD-1 antibody is nivolumab, or pembrolizumab, and the anti-PD-L1 antibody is atezolizumab, avelumab, or durvalumab.

5. The method of claim 1,
wherein the anti-PD-1 antibody comprises:
(i) H-CDR1 comprising the amino acid sequence of SEQ ID NO: 2, H-CDR2 comprising the amino acid sequence of SEQ ID NO: 3, H-CDR3 comprising the amino acid sequence of SEQ ID NO: 4, L-CDR1 comprising the amino acid sequence of SEQ ID NO: 6, L-CDR2 comprising the amino acid sequence of SEQ ID NO: 7, and L-CDR3 comprising the amino acid sequence of SEQ ID NO: 8, or
(ii) H-CDR1 comprising the amino acid sequence of SEQ ID NO: 32, H-CDR2 comprising the amino acid sequence of SEQ ID NO: 33, H-CDR3 comprising the amino acid sequence of SEQ ID NO: 34, L-CDR1 comprising the amino acid sequence of SEQ ID NO: 36, L-CDR2 comprising the amino acid sequence of SEQ ID NO: 37, and L-CDR3 comprising the amino acid sequence of SEQ ID NO: 38.
and wherein the anti-PD-L1 antibody comprises:
(iii) H-CDR1 comprising the amino acid sequence of SEQ ID NO: 42, H-CDR2 comprising the amino acid sequence of SEQ ID NO: 43, H-CDR3 comprising the amino acid sequence of SEQ ID NO: 44, L-CDR1 comprising the amino acid sequence of SEQ ID NO: 46, L-CDR2 comprising the amino acid sequence of SEQ ID NO: 47, and L-CDR3 comprising the amino acid sequence of SEQ ID NO: 48, or
(iv) H-CDR1 comprising the amino acid sequence of SEQ ID NO: 52, H-CDR2 comprising the amino acid sequence of SEQ ID NO: 53, H-CDR3 comprising the amino acid sequence of SEQ ID NO: 54, L-CDR1 comprising the amino acid sequence of SEQ ID NO: 56, L-CDR2 comprising the amino acid sequence of SEQ ID NO: 57, and L-CDR3 comprising the amino acid sequence of SEQ ID NO: 58, or
(v) H-CDR1 comprising the amino acid sequence of SEQ ID NO: 62, H-CDR2 comprising the amino acid sequence of SEQ ID NO: 63, H-CDR3 comprising the amino acid sequence of SEQ ID NO: 64, L-CDR1 comprising the amino acid sequence of SEQ ID NO: 66, L-CDR2 comprising the amino acid sequence of SEQ ID NO: 67, and L-CDR3 comprising the amino acid sequence of SEQ ID NO: 68.

6. The method of claim 1,
wherein the anti-PD-1 antibody comprises:
(i) a variable heavy chain sequence (VH) of SEQ ID NO:1 and a variable light chain sequences (VL) of SEQ ID NO:5, or
(ii) a variable heavy chain sequence (VH) of SEQ ID NO:31 and a variable light chain sequences (VL) of SEQ ID NO:35,
and wherein the anti-PD-L1 antibody comprises:
(iii) a variable heavy chain sequence (VH) of SEQ ID NO:41 and a variable light chain sequences (VL) of SEQ ID NO:45, or
(iv) a variable heavy chain sequence (VH) of SEQ ID NO:51 and a variable light chain sequences (VL) of SEQ ID NO:55, or
(v) a variable heavy chain sequence (VH) of SEQ ID NO:61 and a variable light chain sequences (VL) of SEQ ID NO:65.

7. The method of claim 1,
wherein the anti-PD-1 antibody comprises:
(i) a heavy chain region (HC) of SEQ ID NO: 9 and a light chain region (LC) of SEQ ID NO: 10, or
(ii) a heavy chain region (HC) of SEQ ID NO: 39 and a light chain region (LC) of SEQ ID NO: 40,
and wherein the anti-PD-L1 antibody comprises:
(iii) a heavy chain region (HC) of SEQ ID NO: 49 and a light chain region (LC) of SEQ ID NO: 50, or
(iv) a heavy chain region (HC) of SEQ ID NO: 59 and a light chain region (LC) of SEQ ID NO: 60, or
(v) a heavy chain region (HC) of SEQ ID NO: 69 and a light chain region (LC) of SEQ ID NO: 70.

8. The method of claim 1, wherein the cancer is lung cancer, in particular non-small cell lung cancer, ovarian cancer, mesothelioma, pancreatic cancer, gastric cancer, colorectal cancer, head and neck cancer, bladder cancer, bile duct cancer, breast cancer, cervical cancer, or esophageal cancer.

9. The method of claim 1, wherein at least one of the anti-CEACAM6 antibody, the anti-PD-1 antibody, or an anti-PD-L1 antibody is administered in simultaneous, separate, or sequential combination with one or more pharmaceutical agents.

10. A method of claim 1, wherein the anti-CEACAM6 antibody is in admixture with the anti-PD-1 antibody.

11. (canceled)
